(19) United States
(12) Patent Application Publication
(10) Pub. No.: US 2009/0169581 A1
(43) Pub. Date: Jul. 2, 2009

(54) STABILIZING FORMULATIONS FOR RECOMBINANT VIRUSES

(76) Inventor: Cigarini Sandrine, St Genis les Ollières (FR)

Correspondence Address: MR. REZA YACOOB;AVENTIS PASTEUR LIMITED, 1755 STEELES AVE. WEST, TORONTO, ON, M2R 3T4, CA

(21) Appl. No.: 12/094,302

(22) PCT Filed: Nov. 15, 2006
(86) PCT No.: PCT/CA2006/001855

§ 371 Date: Nov. 4, 2008

Related U.S. Application Data
(60) Provisional application No. 60/597,280, filed on Nov. 21, 2005.

(51) Int. Cl. A61K 39/00; C12N 7/00

(52) U.S. Cl. 424/199.1; 435/235.1

(57) ABSTRACT

This invention relates to preparations of viruses, e.g. for vaccine or other pharmaceutical or research use, to their stabilization, and to processes of producing such preparations, as well as to their use, e.g. as vaccines or as virus vectors. The formulations comprise a sugar, a preservative, a dispersing agent, a thermal stability agent, a buffer, and up to three distinct types of amino acids without impacting the structural appearance of the lyophilized product.

### FIELD OF THE INVENTION

This invention relates to preparations of viruses, e.g. for vaccine or other pharmaceutical or research use, to their stabilization, and to processes of producing such preparations, as well as to their use, e.g. as vaccines or as virus vectors.

### BACKGROUND OF THE INVENTION

Numerous methods are known for producing live virus preparations for vaccine and other purposes. Formulations and methods useful in freezing, lyophilizing, or otherwise storing viable virus preparations for laboratory or vaccine use in order to preserve their activity are also known.

Typically, recombinant viruses have been stored as freeze-dried pellets containing sucrose, hydrolysates of casein and/or collagen in phosphate-buffered physiological saline (PBS). These pellets are then re-hydrated in a pharmaceutically acceptable solution such as 0.4-0.9% NaCl. However, there are significant disadvantages associated with such formulations and are known in the art. Among these are the use of animal-derived substances, incompletely defined components, complex preparation procedures, high cost, and inability to maintain certain desired characteristics of the virus.

There remains a need in the art for formulations suitable for preparing stabilized virus preparations for use in immunological formulations and vaccines. It is desirable to provide stabilizing formulations that preserve desired characteristics of a virus, immunological formulation or vaccine, include virus viability and infectivity. In addition, there is a need in the art for stabilizing formulations that are not derived from animal-based products due to concerns relating to animal-borne diseases. It is further desirable to provide high-titer low volume formulations amenable to rapid freeze-drying treatments. Such formulations, formulations, and methods for using the same are described herein.

### BRIEF DESCRIPTION OF THE DRAWINGS

FIG. 1. Comparison of the liquid formulation and one embodiment of the invention, a new freeze-dried formulation F12 (FD), on the stability of a recombinant viral preparation at various temperatures. Infectious titre (CCID₅₀) of each viral formulation were measured at the indicated times.

### SUMMARY OF THE INVENTION

The present invention provides stabilizing formulations (“stabilizers”) for preserving viruses, such as viral vectors, for various uses including within immunological formulations and vaccines. In one embodiment, the formulation comprises a sugar, a preservative, a dispersing agent, a thermal stability agent, a buffer, and up to three distinct types of amino acids (i.e., one, two or three distinct types of amino acid(s)). In one embodiment, the formulation comprises three distinct types of amino acids. In another embodiment, the formulation comprises two distinct types of amino acids. In another embodiment, the formulation comprises only a type of amino acid. It is preferred that the amino acid(s) is/are arginine, alanine, serine or glycine. It is further preferred that the amino acid(s) is/are arginine, serine or glycine. The virus is added to the stabilizing formulation and retains particular, measurable characteristics (i.e., viability, infectivity) for a desired amount of time. Preferred formulations retain certain desirable and measurable characteristics such as favorable appearance and dissolution times under specific conditions in the presence of a virus, which are described below. Other embodiments of the present invention will be evident from the description, examples and claims shown below.

### DETAILED DESCRIPTION OF THE INVENTION

Typically, recombinant viruses such as the avipox virus ALVAC have been stored as freeze-dried pellets containing sucrose, hydrolysates of casein and/or collagen in phosphate-buffered physiological saline (PBS). These pellets are then re-hydrated in a pharmaceutically acceptable solution such as 0.4-0.9% NaCl. However, there are significant disadvantages associated with such formulations and are known in the art to be difficult to work with. Thus, provided herein are new stabilizing formulations, as described below.

The present invention provides formulations for stably storing and preserving a virus, including a recombinant virus, for use as expression vectors, immunological formulations, and/or vaccines. The formulations are useful in methods of preparing, storing, and using such viruses with greater ease, at a lesser cost, and without a significant decrease in viral activity as compared to presently available formulations. Such formulations may be referred to as “stabilizing formulations” and typically include a sugar (i.e., sucrose or sorbitol, trehalose, saccharose, mannitol, lactose), a preservative (which may be a sugar, amino acid, other component), a dispersing agent (i.e., polyvinyl pyrrolidone 40, dextran, PEG), a thermal stability agent (i.e., urea), a buffer (i.e., Tris, phosphate-buffered saline (PBS), sodium phosphate, acetate, Borate, Hepes, MOPS, PEG) and one or more amino acids.

It is preferred that no more than one, two, three, or four amino acids are included in the formulation. It should be understood by those of skill in the art that the amino acids referred to in describing the composition do not include amino acids found within or released into the formulation from a virus, adjuvant or other component added to the formulation subsequent to its preparation. Thus, the amino acids described as being part of the formulation are present prior to addition of a virus or adjuvant to the formulation.

In a most preferred embodiment, a single amino acid is included in the formulation. In a preferred embodiment, the formulation includes at least one of arginine, serine or glycine. In a more preferred embodiment, the formulation comprises a single amino acid which is arginine, serine or glycine. The amino acid(s) are preferably present in the formulation at or under about 100 mg/ml. More preferably, the amino acid(s) is present in the formulation at about 90-95 mg/ml, about 85-90 mg/ml, about 80-85 mg/ml, or about 80 mg/ml. Individual amino acids are widely available to those of skill in the art.

A stabilizing formulation is preferably used to store a virus as a liquid, freeze-dried preparation, lyophilized preparation, or other form. With respect to the liquid, it is preferred that the liquid formulation is a pharmaceutical formulation. The freeze-dried or lyophilized preparation is typically converted to a liquid form by reconstituting it using a liquid, such as a pharmaceutically acceptable carrier. The pharmaceutically acceptable carrier may be a liquid carrier that contains a buffer and a salt, for instance (i.e., PBS). Examples of suitable buffers and salts, as well as other types of pharmaceutically acceptable carriers, are well known in the art.

In assessing the suitability of a particular formulation for storing a virus preparation, the visual appearance of the formulation has been determined to be an important indicator of suitability. For instance, the most suitable formulations present a smooth, white layer or “cake” which is not retracted from the sides of the vial after lyophilization and storage at about −20° C. for about 52 weeks. Less suitable formulations appear “melted”, “boiled” or otherwise malformed, and retracted from the sides of the vial after storage. As shown in the experimental results presented below, the smooth, white layer is associated with faster dissolution times, which is another desirable characteristic of the formulations described herein. The smooth and white layer cake is strongly associated with a formulation useful for stably preserving a virus.

As discussed above, dissolution time is a very important characteristic of a suitable formulation. It is preferred that, following lyophilization of the virus preparation, the formulation have a dissolution time in a pharmaceutically acceptable carrier such as PBS of about 20-25 seconds, about 15-20 seconds or, preferably, about 15 seconds or less after storage for about 52 weeks at about 5° C. This provides the skilled artisan with a formulation that is rapidly useable in the field.

The temperature at which the virus is maintained in the stabilizing formulation is any suitable for maintaining the virus/formulation in a desired state (i.e., determined by observing appearance, dissolution time, titre or other characteristic of the preparation) over the time period of storage (i.e., up to about 52 weeks). The formulation is typically and most conveniently maintained at a temperature below about 10° C., (i.e., about 5° C.). In certain situations, the formulation will be maintained at −20° C.

A suitable pH for the formulation following reconstitution is any pH at which the virus is maintained in a desired state (i.e., viability, infectious titer, dissolution time) over the time period of storage (i.e., up to about 52 weeks). For example, the pH of the liquid formulation desirably is about 6-9, 6-8.5, 6.5-8.5, 7-8.5, 7.5-8.5, 6-8, 6.5-8, 7-8, 7.5-8, or 7-7.5. It is preferred that the formulation have a pH of about 7.5. The liquid formulation can be placed (e.g., maintained or stored) in any suitable container. Typically, the container will comprise, consist essentially of, or consist of glass or plastic in the form of a vial or other storage container.

Many different viruses may be utilized in practicing the present invention. Suitable viruses include, for example, Adenoviruses, Arboviruses, Astroviruses, Bacteriophages, Enteroviruses, Gastroenteritis Viruses, Hantavirus, Coxsackie viruses, Hepatitis A Viruses, Hepatitis B Viruses, Hepatitis C Viruses, Herpesviruses (for example, Epstein Barr Virus (EBV), Cytomegalovirus (CMV) and Herpes Simplex Virus (HSV)), Influenza Viruses, Norwalk Viruses, Polio Viruses, Chordopoxyiridae (i.e., 5 Orthopoxvirus, vaccinia, MVA, NYVAC, Avipoxvirus, canarypox, ALVAC, ALVAC(2), fowlpox, Rhabdoviruses, Reoviruses, Rhinoviruses, Rotavirus, Retroviruses, Baculoviridae, Caliciviridae, Caulimoviridae, Coronaviridae, Filoviridae, Flaviviridae, Hepadnaviridae, Nodaviridae, Orthomyxoviridae, Paramyxoviridae, Papovaviridae, Parvoviridae, Phycodnaviridae, Picornaviridae, and Togaviridae, and modified viruses originating from, based upon, or substantially similar to any of the foregoing or other suitable virus. Preferred viruses for use in practicing the present invention are poxvirases, in particular ALVAC. Other suitable viruses are known in the art as described in, for example, Fields et al., Virology (34th ed., Lippincott Williams & Wilkins (2001)).

In certain embodiments, the recombinant virus contains within its genome nucleic acid sequence encoding an antigen or immunogen, such that the virus may be used in an immunological formulation or vaccine. The term “recombinant virus” refers to any virus having inserted into the viral genome a heterologous gene that is not naturally part of the viral genome. An immunological formulation is one that, upon administration to a host, results in an immune response directed or reactive to the antigen or immunogen encoded by the virus. This immune response may or may not be protective or provide immunity to the host. A vaccine is a formulation that causes the host to develop a protective immune response directed or reactive to the antigen or immunogen encoded by the host. Immune responses may be measured by any of the many techniques available to one of skill in the art, including but not limited to ELISA, BIACORE, DOT-BLOT, immunodiffusion techniques.

In certain cases, the recombinant virus may encode one or more tumor antigens (“TA”). TA includes both tumor-associated antigens (TAAs) and tumor-specific antigens (TSAs), where a cancerous cell is the source of the antigen. A TAA is an antigen that is expressed on the surface of a tumor cell in higher amounts than is observed on normal cells or an antigen that is expressed on normal cells during fetal development. A TSA is an antigen that is unique to tumor cells and is not expressed on normal cells. TA further includes TAAs or TSAs, antigenic fragments thereof, and modified versions that retain their antigenicity. TAs are typically classified into five categories according to their expression pattern, function, or genetic origin: cancer-testis (CT) antigens (i.e., MAGE, NY-ESO-1); melanocyte differentiation antigens (i.e., Melan A/MART-1, tyrosinase, gp100); mutational antigens (i.e., MUM-1, p53, CDK-4); overexpressed ‘self’ antigens (i.e., HER-2/neu, p53); and, viral antigens (i.e., HPV, EBV). For the purposes of practicing the present invention, a suitable TA is any TA that induces or enhances an anti-tumor immune response in a host to whom the TA has been administered. Suitable TAs include, for example, gp100 (Cox et al., Science, 264:716-719 (1994)), MART-1/Melan A (Kawakami et al., J. Exp. Med., 180:347-352 (1994)), gp75 (TRP-1) (Wang et al., J. Exp. Med., 186:1131-1140 (1996)), tyrosinase (Wolfel et al., Eur. J. Immunol., 24:759-764 (1994); WO 200175117; WO 200175016; WO 200175007), NY-ESO-1 (WO 98/14464; WO 99/18206), melanoma proteoglycan (Hellstrom et al., J. Immunol., 130:1467-1472 (1983)), MAGE family antigens (i.e., MAGE-1, 2, 3, 4, 6, and 12; Van der Bruggen et al., Science, 254:1643-1647 (1991); U.S. Pat. No. 6,235,525), BAGE family antigens (Boel et al., Immunity, 2:167-175 (1995)), GAGE family antigens (i.e., GAGE-1,2; Van den Eynde et al., J. Exp. Med., 182:689-698 (1995); U.S. Pat. No. 6,013,765), RAGE family antigens (i.e., RAGE-1; Gaugler et al., Immunogenetics, 44:323-330 (1996); U.S. Pat. No. 5,939,526), N-acetylglucosaminyltransferase-V (Guilloux et al., J. Exp. Med., 183:1173-1183 (1996)), p15 (Robbins et al., J. Immunol. 154:5944-5950 (1995)), β-catenin (Robbins et al., J. Exp. Med., 183:1185-1192 (1996)), MUM-1 (Coulie et al., Proc. Natl. Acad. Sci. USA, 92:7976-7980 (1995)), cyclin dependent kinase-4 (CDK4) (Wolfel et al., Science, 269:1281-1284 (1995)), p21-ras (Fossum et al., Int. J. Cancer, 56:40-45 (1994)), BCR-abl (Bocchia et al., Blood, 85:2680-2684 (1995)), p53 (Theobald et al., Proc. Natl. Acad. Sci. USA, 92:11993-11997 (1995)), p185 HER2/neu (erb-β1; Fisk et al., J. Exp. Med., 181:2109-2117 (1995)), epidermal growth factor receptor (EGFR) (Harris et al., Breast Cancer Res. Treat, 29:1-2 (1994)), carcinoembryonic antigens (CEA) (Kwong et al., J. Natl. Cancer Inst., 85:982-990 (1995) U.S. Pat. Nos. 5,756,103; 5,274,087; 5,571,710; 6,071,716; 5,698,530; 6,045,802; EP 263933; EP 346710; and, EP 784483); carcinoma-associated mutated mucins (i.e., MUC-1 gene products; Jerome et al., J. Immunol., 151:1654-1662 (1993)); EBNA gene products of EBV (i.e., EBNA-1; Rickinson et al., Cancer Surveys, 13:53-80 (1992)); E7, E6 proteins of human papillomavirus (Ressing et al., J. Immunol, 154:5934-5943 (1995)); prostate specific antigen (PSA; Xue et al., The Prostate, 30:73-78 (1997)); prostate specific membrane antigen (PSMA; Israeli, et al., Cancer Res., 54:1807-1811 (1994)); idiotypic epitopes or antigens, for example, immunoglobulin idiotypes or T cell receptor idiotypes (Chen et al., J. Immunol., 153:4775-4787 (1994)); KSA (U.S. Pat. No. 5,348,887), kinesin 2 (Dietz, et al. Biochem Biophys Res Commun 2000 Sep. 7; 275(3):731-8), HIP-55, TGF β-1 anti-apoptotic factor (Toomey, et al. Br J Biomed Sci 2001; 58(3):177-83), tumor protein D52 (Bryne J. A., et al., Genomics, 35:523-532 (1996)), H1FT, NY-BR-1 (WO 01/47959), NY-BR-62, NY-BR-75, NY-BR-85, NY-BR-87, NY-BR-96 (Scanlan, M. Serologic and Bioinformatic Approaches to the Identification of Human Tumor Antigens, in Cancer Vaccines 2000, Cancer Research Institute, New York, N.Y.), BCY1, BFA4, BCA4, and BFY3, including “wild-type” (i.e., normally encoded by the genome, naturally-occurring), modified, mutated versions as well as other fragments and derivatives thereof. Any of these TAs may be utilized alone or in combination with one or more other TAs in a co-immunization protocol.

In other cases, the recombinant virus may encode an antigen or immunogen derived from an pathogenic organism. Exemplary infectious disease agents include bacteria, viruses, fungi, parasites, and the like. Particular exemplary infectious agents include Bacillus spp. (i.e., B. anthracis), Bordetella spp. (i.e., B. brochiseptica, B. parapertussis, B. pertussis), Borellia (i.e., B. burgdorferi), Brucella spp., Campylobacter spp., Chalmydia spp. (i.e., C. trachomatis), Clostridium spp. (i.e., Clostridium botulinum), Corynebacterium (i.e., C. diphtheria), Enterobacter spp., Escherichia spp. (i.e., E. coli), Haemophilus spp. (i.e., H. influenzae), Helicobacter spp. (i.e., H. pylori), Klebsiella spp., Legionella spp., Listeria spp., Mycobacterium spp. (i.e., M. tubercolosis), Mycoplasma spp., Neisseria spp. (i.e., N. meningitidis, N. gonnorhoeae), Nocardia spp., Pasteurella spp., Proteus spp., Rickettsia spp., Salmonella spp. (i.e., S. entiriditis, S. typhi), Shigella spp. (i.e., Shigella flexneri), Staphylococcus spp. (i.e., S. aureus), Streptococcus spp. (i.e., S. pneumoniae), Vibrio spp. (i.e., V. cholerea), Coronavirus, CMV, Dengue virus, Ebola virus, EBV, Hepatitis virus (i.e., Hepatitis A, B, C, D, and E), Herpes virus, HIV, Influenza virus, Measles virus, Mumps virus, Papillomaviruses (human), pox viruses (i.e., vaccinia, smallpox), polio virus, rabies virus, RSV, West Nile virus, Yellow Fever virus, Aspergillus spp., Blastomyces spp., Candida spp., Coccidioides spp., Cryptococcus spp., Histoplasma spp., Coccidia spp., Cryptosporidum spp., Entamoeba spp. (i.e., E. histolytica), Giardia spp. (i.e., Giardia lamblia), Leshmania spp., Plasmodium spp., Schistosoma spp., Toxoplasma spp. (i.e., Toxoplasma gondii), Trichinella spp., and Trypanosoma spp., among others.

In certain embodiments, an immunogenic formulation or vaccine of the present invention may be administered in combination with one or more adjuvants to boost the immune response. Exemplary adjuvants are shown in Table 1 below:

TABLE 1
Types of Immunologic Adjuvants

| Type of Adjuvant | General Examples | Specific Examples/References |
|---|---|---|
| 1 Gel-type | Aluminum | (Aggerbeck and Heron, 1995) |
| | hydroxide/phosphate (“alum | |
| | adjuvants”) | |
| | Calcium phosphate | (Relyveld, 1986) |
| 2 Microbial | Muramyl dipeptide (MDP) | (Chedid et al., 1986) |
| | Bacterial exotoxins | Cholera toxin (CT), E. coli labile |
| | | toxin (LT)(Freytag and Clements, |
| | | 1999) |
| | Endotoxin-based adjuvants | Monophosphoryl lipid A (MPL) |
| | | (Ulrich and Myers, 1995) |
| | Other bacterial | CpG oligonucleotides (Corral |
| | | and Petray, 2000), BCG |
| | | sequences (Krieg, et al. Nature, |
| | | 374: 576), tetanus toxoid (Rice, et |
| | | al. J. Immunol., 2001, 167: |
| | | 1558-1565) |
| 3 Particulate | Biodegradable | (Gupta et al., 1998) |
| | polymer microspheres | |
| | Immunostimulatory complexes | (Morein and Bengtsson, 1999) |
| | (ISCOMs) | |
| | Liposomes | (Wassef et al., 1994) |
| 4 Oil-emulsion | Freund's incomplete adjuvant | (Jensen et al., 1998) |
| and surfactant- | | |
| based adjuvants | | |
| | Microfluidized emulsions | MF59 (Ott et al., 1995) |
| | | SAF (Allison and Byars, 1992) |
| | | (Allison, 1999) |
| | Saponins | QS-21 (Kensil, 1996) |
| 5 Synthetic | Muramyl peptide derivatives | Murabutide (Lederer, 1986) |
| | | Threony-MDP (Allison, 1997) |
| | Nonionic block copolymers | L121 (Allison, 1999) |
| | Polyphosphazene (PCPP) | (Payne et al., 1995) |
| | Synthetic polynucleotides | Poly A:U, Poly I:C (Johnson, |
| | | 1994) |

The present invention is further described in the following examples. The examples serve only to illustrate the invention and are not intended to limit the scope of the invention in any way.

### EXAMPLES

### Virus Stabilizing Formulation—Materials

The following chemicals, filters, and vials were utilized for the experiments described below: Urea (Lot Number 101K0040 Sigma); L.Ala Powder (Lot Number 042K0900 Sigma); MSG (Lot Number 91K0096 Sigma); L. Arg Powder (Lot Number 42K0183 Sigma); EAA (Lot Number 3065605 Gibco); Glycine Powder (Lot Number 32K2502 Sigma); Tris (Lot Number 73378B BioRad); L.Ser Powder (Lot Number 111K0883 Sigma); D Mannitol (Lot Number 22K0111 Sigma); NEAA (Lot Number 3065603 Gibco); NaCl (BDH Lot Number 12833/MO89160); Concentric HCl (Lot Number 299102 BDH); Sucrose (Lot Number 51K0026); Sucrose (Lot Number 022K0065 Sigma, Lot Number K27819853/0076535B Merck kGAA); PVP₄₀ (Lot Number 120K0117, Lot Number 71K0064 Sigma); Sorbitol (Lot Number 51K0005 Sigma, Lot Number 042K01351 Sigma); Glutamic Acid (Lot Number 91K0096 Sigma); Vials (BV0030, 3 ml White Tubular Glass Vial); Stopper (3101820, 13 mm V-32 4432/50 Gray Butyl Serum Stopper Latex free); Seals (CS0001, 13 mm One piece Aluminium Seal); Filter 0.2 μm (Lot Number 476291 Nalgene® AC, Lot Number M2MN00586 ZapCap®). ALVAC Virus vCP307 encoding the HIV antigen gp120, Lot Number PX-0246 and Lot Number PX-0230) was utilized as the exemplary virus for the experiments described below.

Methods—pH

As manipulations such as freeze-drying and storage can alter pH, determining the pH of the ALVAC freeze-dried formulations is an important measure of stability. The pH meter (VWR Scientific Products SB301, symphony) is calibrated with standard pH buffer (Orion Application Solutions, pH Buffers 7.00, 10.01 and 4.01), which span the expected pH range of the sample. A fresh portion of the sample is placed in a test tube and the electrode is immersed in it. When the digital display shows a constant reading, the reading is recorded to two decimal places.

Osmolality

Osmolality is the total solute concentration of an aqueous solution. Osmometers measure the number of solute particles irrespective of molecular weight or ionic charge. This study used the Advanced Micro-Osmometer Model 3300, which relies on freezing-point depression to measure osmolality. The osmometer was calibrated using 50 mOsm/kg and 850 mOsm/kg Calibration Standards as per manufacturing instructions. Calibration was verified by running Clinitrol 290 reference Solution. 20 μL samples were loaded into the plunger and inserted into the osmometer sample port for measurement. When the digital display shows a constant reading, the reading is recorded.

Residual Moisture

Residual moisture (RM) is the amount of bound water that remains in a freeze-dried product following primary drying. The Karl Fisher Technique for testing for residual moisture, used in this study, determines water content by volumetric titration. This is measured as the weight percentage of water remaining compared to the total weight of the dried product. RM is an indicator of stability as exposure to moisture during storage can destabalize a product. The European Pharmacopea (V Edition) recommends an RM below 3%. This RM helps avoid microorganism development as well as preventing chemical and physical degradation. The Karl Fisher Coulometric Method is used with a test method designed for use with the Mitsubishi, Model CA-06 Automatic Titration system which determines the end point amperometrically. It is operated in a dry box which is maintained at less than 15% relative humidity with phosphorus pentoxide and a constant flow of dry air at ˜5 mL/min with a temperature range of 20 to 25° C. Weighings are made on a Sartorius balance located in the dry box.

Infectious Titer (CCID₅₀)

CCID₅₀ is a technique used to determine the titre (infectivity) of a virus, in this case, titre of the freeze-dried ALVAC formulation following dissolution. Titre is reflected by the dilution of a virus required to infect 50% of a given batch of inoculated cell cultures. The assay relies on the presence and detection of cytocidal virus particles (those capable of causing a cytopathic effect (CPE)). Host cells are grown in confluent healthy monolayers, typically in 96-well plates, to which aliquiots of virus dilutions are added. On incubation, the virus replicates and progeny virions are released, which in turn infect healthy cells. The CPE is allowed to develop over a period of time, and wells are scored for the presence or absence of CPE. The method becomes more accurate with increasing number of wells per dilution. This test is crucial for determining the loss of activity of the attenuated ALVAC virus during storage.

Samples of ALVAC viral freeze-dried product were serially titrated onto 96-well plates according to SOP Number 22 PD-039 v.1.0. A suspension of QT35 (quail) cells was added to each 96-well plate. Following a six-day incubation period at 36° C.±1° C., the wells showing cytopathic effect (CPE) were counted. The concentration of the virus in the product was calculated using the least squares method and expressed as the number of 50% infective doses per ml of product.

Freeze-Drying Cycle

Lyophilization is a dehydration technique in which a dry state is achieved by freezing a wet substance and evaporating the resulting ice under vacuum through a sublimation process (without melting). This process is conventionally divided into three stages: pre-freezing, primary or sublimation drying and secondary or desorption drying. The 24 hours freeze-drying cycle of ALVAC-based expression vectors was run as summarized in Table 2 below:

TABLE 2
Freeze Dryer Cycle

| Steps | Parameters | Programmable Values | |
|---|---|---|---|
| Freezing (3 Hours) | Loading Temperature | 5° | C. |
| | Temperature at the end | −40° | C. |
| | of Freezing | | |
| Sublimation (Primary | Pressure | 70 | μBar |
| Drying) (16 Hours, 30 | Time to Rise to Shelf | 1 | Hour |
| Minutes) | Temperature | | |
| | Shelf Temperature | −17° | C. |
| | Duration | 13 | Hours |
| | Time to Rise to Shelf | 2 Hours, 30 Minutes | |
| | Temperature | | |
| Desorption (Secondary | Shelf Temperature | 45° | C. |
| Drying) (4 Hours) | Product Temperature to | 25° | C. |
| | Reestablish an Effective | | |
| | Vacuum | | |

| | | |
|---|---|---|
| Stoppering | Under Nitrogen | Under Nitrogen |

The qualitative and quantitative contributions of various components in a stabilizing formulation, such as: amino acids, sugars (sucrose, sorbitol, mannitol) and polymers (poly-vinyl-pyrolidone (PVP)) was evaluated in different stabilizer formulation. The components and concentrations are shown in Table 3. Formulation stability was then assessed using the following assays: appearance, dissolution time, appearance post-dissolution, power of hydrogen (pH), residual moisture and, infectivity (CCID₅₀).

Each stabilizing formulation was prepared under laminar flow conditions. The pH of each formulation was adjusted to be between about 7.2 and about 7.4 for each formulation. Each formulation was filtered through a 0.2 μm poly-vinyl di-fluoride (PVDF) disposable filter, labeled, assigned a lot number and then stored at 5° C. until the lyophilization.

One day prior to freeze-drying, viral ALVAC crude harvest (in 10 mM Tris (pH 9.0) buffer) was thawed in a water bath at 30° C.±2° C. Two dilutions (1/2 and 1/6) were made from the crude harvest to prepare 120 ml of Final Bulk Product (FBP). The first dilution was prepared using the concentrated stabilizer while the second dilution was prepared using a 1:2 dilution of stabilizer with water for injection (WFI).

Set up for the freeze-drying process involved a number of steps. First, 400 vials per formulation were filled with 0.3 mL of FBP in an isolator using 3 ml US glass vials. Second, all of the vials were loaded in a tray on four different shelves and current freeze-drying stabilizers, PO6 and PO7, were run as the Controls. To prevent collapsing, each tray was located in the middle of each shelf and left inside in order to avoid direct contact with shelves. Placing the vials that contained thermocouples in the front of the chamber enabled monitoring of the temperature of the product. Finally, the adapted 24 hours ALVAC lyophilization cycle was performed (see Material and Methods).

Following the run, vials were unloaded and stopped with Poly-Vinyl-Carbon (PVC) stoppers as well as, hermetically capped with Alu-Alu caps. During unloading, the cake aspect of vials from the edge, the middle, the front and the back of the tray was observed for homogenicity. The freeze-dryer graph was analyzed to ensure the cycle carried out each step without experiencing complications. Vials were then stored at −20° C. for further sampling and testing.

The effect of formulation on the stability of freeze-dried protein was investigated using both real time and accelerated stability studies. Each assay was performed on each set of five samples at various time points and various temperatures (−20° C., 2-8° C., 35-39° C., at each of weeks 1-6, 8, 12, 26 and 52). The following characteristics of each sample was then recorded: Appearance of Lyophilizate, Dissolution Time, Appearance following Dissolution, pH and Infectious Titer (CCID₅₀).

Further stability studies were then conducted using those formulations having the most desirable characteristics. Each assay was performed on each set of four to six samples at various time points and various temperatures (−20° C., 2-8° C., 37° C., and 45° C., at each of days 0, 3, 7, 11, 14, 21, 25, 28, 35, 56, 84, 182, and 364). The following characteristics of each sample was then recorded: Appearance of Lyophilizate, Dissolution Time, Appearance following Dissolution, pH, Residual Moisture and Infectious Titer (CCID₅₀). An additional observation point was added at 45 W was added for Control, F12 (n=6).

A final stability study of the freeze-dried ALVAC Formulations F12 (extra time point samples from the earlier studies) was performed at 23-27° C. on the set of vials at weeks 1, 3, 5, 8 and 12 (n=6). The formulations were then assessed for Infectious Titer (CCID₅₀), pH, Reconstitution Time, Appearance and Osmolality.

Stabilizing Formulations

Several new stabilizing formulations were developed and tested. The components included in each such formulation are shown in Table 3. It should be noted that particular care must be taken when adding PVP40 into the formulation. The stirring speed should be increased for a certain amount of time and then decreased when the PVP40 dissolves. If the speed is not increased, undesired aggregates will typically appear. Time and speed for mixing depend on the prepared volume, as would be understood by one of skill in the art.

Experimental Results

Appearance of the Lyophilizate

Following 52 weeks of storage at −20° C. and 5° C., the appearance of the lyophilizate for the different formulations was assessed. The appearance of the cake for formulations F9, F10, F11 and F12 in the first set of experiments met with the required specifications (smooth, white layer that is not retracted from the edges of the vial). These required “cake” appearance specifications indicate that the formulation is able to maintain the physico-chemical integrity of the virus by protecting it and the surrounding environment from an eventual collapse or melting. The collapse of the cake could be due to an aggressive freeze-dried process or storage conditions such as long term storage or high temperature. It has been determined that a formulation having the proper cake appearance provides an environment suitable for virus stabilization. It was determined that the mixture of the 20 essential and non-essential amino acids used previously could be replaced by L-Arginine, L-Alinine, Serine and/or Glycine at high concentrations without impacting the structural appearance of the lyophilizate.

Several other formulations had appeared boiled/melted, retracted from the edge of the vials, and presented a non-homogenous appearance (i.e., not a smooth, white layer); as such, these were rejected for failing to meet the appearance criteria. As discussed below, this appearance was associated with an undesirable increased dissolution time. Each of these rejected formulations lacked a dispersing agent(s) (i.e., polyvinyl pyrrolidone (PVP40), sorbitol), thus demonstrating the significant impact of such components on the structure of the cake. The cake, for these formulations appeared loose or retracted from the edge of the vials and consequently, was also rejected based on the appearance criteria.

Dissolution Time, Appearance after Dissolution, pH and Osmolality

Dissolution time for formulations F9 was slightly increased following 52 weeks of storage at −20° C. and 5° C. compared with the other stabilizers (F10-12), which dissolved in less than 15 seconds. Each vial was reconstituted with 1 ml of NaCl 0.4% and mixed manually until dissolution of the entire cake. It was concluded that the inclusion of PVP40 positively effects on the dissolution time of the final product. For those samples stored under stress conditions (35-37° C.), the dissolution time was considerably increased for all of the formulations, from 15 seconds to more than one minute. Furthermore, where the appearance of the cake was melted, the product stuck to the vials making it difficult to reconstitute the lyophilizate.

For all of the formulations under all tested conditions, the pH remained stable between 7.5±0.5 units.

For formulations F12 osmolality ranged between 350±100 mOsm/kg.

Residual Moisture (RM)

Formulations F12 the Residual Moisture results were <3% and compliant to the recommendations of the European Pharmacopea (V Edition). The other formulations were not tested.

TABLE 4
Residual Moisture - Formulations F12

| | | Water | | |
|---|---|---|---|---|
| | | First Run | Second Run | Third Run |
| Formulation | Sample | CA-03-031 | CA-03-044 | CA-03-059 |
| F12 | 1 | 1.47 | 1.19 | .99 |
| | 2 | 1.34 | 1.20 | 1.08 |
| | 3 | 1.48 | 1.25 | 1.02 |

Infectious Titer

A significant decrease in viral activity is considered a drop in activity of more than about 10-20%. All formulations seemed to maintain the infectious titer, following 52 weeks of storage at −20° C. and 2-8° C., with no significant loss observed. All of the formulations tested were similar to the Control confirming the precision of the method (±0.3 log 10 CCID50/ml). At stress conditions, infectious titer of the Control and of the tested formulations was maintained for the full 8 weeks of storage at 35-37° C.

Stability of Liquid Vs. Freeze-Dried Formulations

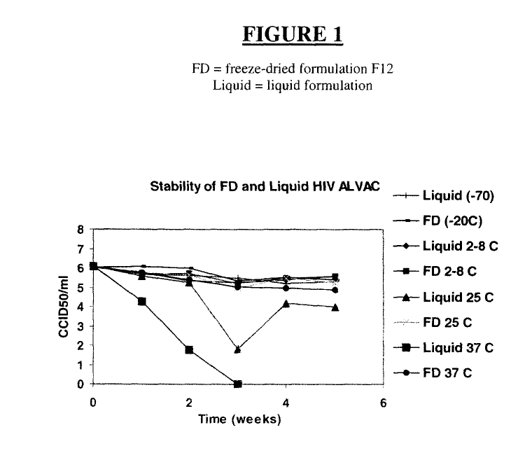

Given the above-described results, F12 (freeze-dried) was selected for comparison to the liquid formulation (ALVAC in 10 mM Tris-HCl, 0.9% NaCl, pH9.0). These formulations were compared for stability of titre in both “real time” (2-8° C.) and under stress or accelerated conditions (23-25° C. and 35-39° C.) and compared with control conditions (−70° C. and −20° C.). Both formulations were prepared with equal volume and equal quantities of ALVAC clarified harvest. Titre was measured using the CCID₅₀ assay. As shown in FIG. 1, freeze-dried F12 showed similar titres as the liquid formulation at 2-8° C. (as well as control temperatures −20° C. and −70° C.) and superior titres to the liquid formulation at 25° C. and 37° C. Given the fact that F12 is a freeze-dried formulation, which is the formulation format favored by those of skill in the art, it can be concluded that F12 is superior to the previously available liquid formulation.

The studies described herein in that a dispersing agent such as PVP40 and/or sorbitol are essential to maintaining the appearance of the freeze-dried formulation (i.e., the “cake”), which is functionally predictive of desired dissolution properties. In addition, it is apparent that the previously utilized mixture of the 20 essential and non-essential amino acids may be replaced by L-Arginine, L-Alanine, Serine and/or Glycine at high concentrations without impacting the structural appearance of the lyophilizate while maintaining infectious titer. This is especially significant in that it simplifies the formulation preparation process and reduces costs associated therewith. Results based on the infectious titer data did not indicate any significant difference amongst the various formulations. All of the formulations were able to maintain an infectious titer for 52 weeks at 2-8° C.

While the present invention has been described in terms of the preferred embodiments, it is understood that variations and modifications will occur to those skilled in the art. Therefore, it is intended that the appended claims cover all such equivalent variations that come within the scope of the invention as claimed.

TABLE 3
Qualitative and Quantitative Formulations

| | | | | | | | | | | | | | | 52-week | | |
|---|---|---|---|---|---|---|---|---|---|---|---|---|---|---|---|---|
| | | | | | | | | | | | | | | Dissolution | | |
| Formu- | | | Su- | Sor- | | | | | Man- | L. | L. | L. | | Time @ | Appearance | |
| lation | EAA | NEAA | crose | bitol | MSG | PVP40 | Tris | Urea | nitol | Arg | Ala | Ser | Gly | 5° C. | −20° C. | 5° C. |
| Control | 40 | 40 | 10 | 10 | 1 | 2 | 1.452 | 1 | 0 | 0 | 0 | 0 | 0 | 5-12 | White layer, | Retracting or |
| | | | | | | | | | | | | | | | smooth cake | White layer, |
| | | | | | | | | | | | | | | | | smooth cake |
| F9 | 0 | 0 | 10 | 10 | 1 | 2 | 1.452 | 1 | 0 | 0 | 80 | 0 | 0 | 29 | White layer, | White layer, |
| | | | | | | | | | | | | | | | smooth cake | smooth cake |
| F10 | 0 | 0 | 10 | 10 | 1 | 2 | 1.452 | 1 | 0 | 80 | 0 | 0 | 0 | 15 | White layer, | White layer, |
| | | | | | | | | | | | | | | | smooth cake | smooth cake |
| F11 | 0 | 0 | 10 | 10 | 1 | 2 | 1.452 | 1 | 0 | 0 | 0 | 80 | 0 | 15 | White layer, | Retracting |
| | | | | | | | | | | | | | | | smooth cake | |
| F12 | 0 | 0 | 10 | 10 | I | 2 | 1.452 | 1 | 0 | 0 | 0 | 0 | 80 | 15 | White layer, | White layer, |
| | | | | | | | | | | | | | | | smooth cake | smooth cake |

What is claimed is:

1. A formulation for maintaining a virus in a stable form, the formulation comprising a sugar, a preservative, a dispersing agent, a thermal stability agent, a buffer, and up to three distinct types of amino acids.

2. A formulation for maintaining a virus in a stable form, the formulation comprising a sugar, a preservative, a dispersing agent, a thermal stability agent, a buffer, and up to two distinct types of amino acids.

3. A formulation for maintaining a virus in a stable form, the formulation comprising a sugar, a preservative, a dispersing agent, a thermal stability agent, a buffer, and a single type of amino acid.

4. The formulation of any one of claims 1-3 wherein the sugar is sucrose or sorbitol.

5. The formulation of any one of claims 1-4 wherein the buffer is Tris.

6. The formulation of any one of claims 1-5 wherein the dispersing agent is polyvinyl pyrrolidone.

7. The formulation of any one of claims 1-6 wherein the dispersing agent is polyvinyl pyrrolidone 40.

8. The formulation of any one of claims 1-7 wherein the amino acid is selected from arginine, serine, and glycine.

9. The formulation of claim 8 wherein the amino acid is arginine.

10. The formulation of claim 8 wherein the amino acid is serine.

11. The formulation of claim 8 wherein the amino acid is glycine.

12. The formulation any one of claims 8-11 wherein the amino acid is present in the formulation at a concentration of less than about 100 mg/ml.

13. The formulation of any one of claims 12 wherein the amino acid is present in the formulation at a concentration of about 80 mg/ml.

14. The formulation of any one of claims 1-13 having a dissolution time of 15 seconds or less at 5° C.

15. The formulation of any one of claims 1-13 appearing upon lyophilization as a smooth, white layer of material after storage at about −20° C. for about 52 weeks.

16. The formulation of any one of claims 1-13 having a dissolution time of 15 seconds or less at 5° C. and appearing upon lyophilization as a smooth, white layer of material after storage at about −20° C. for about 52 weeks.

17. The formulation of any one of claims 1-16, further comprising a recombinant virus.

18. The formulation of claim 17 wherein the virus is selected from the group consisting of adenovirus, influenza virus, pox virus, and retrovirus.

19. The formulation of claim 18 wherein the virus is a pox virus.

20. The formulation of claim 19 wherein the poxvirus is an orthopox virus or an avipox virus.

21. The formulation of claim 20 wherein the orthopox virus is selected from the group consisting of vaccinia, MVA, and NYVAC.

22. The formulation of claim 20 wherein the avipox virus is selected from the group consisting of canarypox, fowlpox, TROVAC, ALVAC and ALVAC(2).

23. The formulation of any one of claims 17-22 wherein the recombinant virus includes within its genome at least one nucleic acid sequence encoding an antigen related to a pathogenic organism or cancer.

24. The formulation of claim 23 wherein the pathogenic organism is selected from the group consisting of Bacillus spp., Bordetella spp., Borellia spp., Brucella spp., Campylobacter spp., Chlamydia spp., Clostridium spp., Corynebacterium, Enterobacter spp., Escherichia spp., Haemophilus spp., Helicobacter spp., Klebsiella spp., Legionella spp., Listeria spp., Mycobacterium spp., Mycoplasma spp., Neisseria spp., Nocardia spp., Pasteurella spp., Proteus spp., Rickettsia spp., Salmonella spp., Shigella spp., Staphylococcus spp., Streptococcus spp., Vibrio spp., Coronavirus, CMV, Dengue virus, Ebola virus, EBV, Hepatitis virus, Herpes virus, HIV, Influenza virus, Measles virus, Mumps virus, Papillomavirus, pox virus, polio virus, rabies virus, RSV, West Nile virus, Yellow Fever virus, Aspergillus spp., Blastomyces spp., Candida spp., Coccidioides spp., Cryptococcus spp., Histoplasma spp., Coccidia spp., Cryptosporidum spp., Entamoeba spp., Giardia spp., Leshmania spp., Plasmodium spp., Schistosoma spp., Toxoplasma spp., Trichinella spp., and Trypanosoma spp.

25. The formulation of claim 24 wherein the infectious agent is selected from the group consisting of B. anthracis, B. brochiseptica, B. parapertussis, B. pertussis, B. burgdorferi, C. trachomatis, Clostridium botulinum, C. diphtheria, E. coli, H. influenzae, H. pylori, M. tubercolosis, N. meningitidis, N. gonnorhoeae, S. entiriditis, S. typhi, Shigella flexneri, S. aureus, S. pneumoniae, V. cholerea, Hepatitis A, Hepatitis B, Hepatitis C, Hepatitis D, Hepatitis E, vaccinia virus, smallpox virus, E. histolytica, Giardia lamblia, and Toxoplasma gondii.

26. The formulation of claim 23 wherein the cancer is selected from the group consisting of cancer of the colon, rectum, breast, skin, lung, brain, and blood.

27. The formulation of claim 23 wherein the nucleic acid sequence encodes a tumor antigen.

28. The method of claim 27 wherein the tumor antigen is selected from the group consisting of gp100, MART-1/Melan A, gp75 (TRP-1), tyrosinase, melanoma proteoglycan, a MAGE family antigen, MAGE-1, MAGE-2, MAGE-3, MAGE-4, MAGE-6, MAGE-12, a BAGE family antigen, a GAGE family antigens, GAGE-1, GAGE-2, a RAGE family antigen, RAGE-1, N-acetylglucosaminyltransferase-V, p15, β-catenin, MUM-1, cyclin dependent kinase-4, p21-ras, BCR-abl, p53, p185 HER2/neu, epidermal growth factor receptor, carcinoembryonic antigen (CEA), a carcinoma-associated mutated mucin, a MUC-1 gene products, an Epstein-Barr Virus EBNA gene product, papillomavirus E7, papillomavirus E6, prostate specific antigen (PSA), prostate specific membrane antigen (PSMA), an idiotypic antigens, KSA, kinesin 2, HIP-55, TGF β-1 anti-apoptotic factor, tumor protein D52, H1FT, NY-BR-1, NY-BR-62, NY-BR-75, NY-BR-85, NY-BR-87, NY-BR-96, BCY1, BFA4, BCY3, BCZ4, fragments thereof, and derivatives thereof.

29. An immunological formulation comprising a formulation of any one of claims 17-28.

30. The immunological formulation of claim 29 further comprising an adjuvant.

31. A vaccine for human or animal use comprising a formulation of any one of claims 17-28.

32. The vaccine of claim 31 further comprising an adjuvant.

33. The formulation of any one of claims 17-32, wherein the formulation is freeze-dried or lyophilized.

34. A method for treating or preventing a disease condition comprising reconstituting in a physiologically acceptable buffer a lyophilized formulation of any one of claims 17-33 and administering the formulation to a host.

35. The method of claim 34 wherein the disease condition is cancer or is caused by a pathogenic organism.

36. The method of claim 35 wherein the species of the pathogenic organism is selected from the group consisting of Bacillus spp., Bordetella spp., Borellia spp., Brucella spp., Campylobacter spp., Chalmydia spp., Clostridium spp., Corynebacterium, Enterobacter spp., Escherichia spp., Haemophilus spp., Helicobacter spp., Klebsiella spp., Legionella spp., Listeria spp., Mycobacterium spp., Mycoplasma spp., Neisseria spp., Nocardia spp., Pasteurella spp., Proteus spp., Rickettsia spp., Salmonella spp., Shigella spp., Staphylococcus spp., Streptococcus spp., Vibrio spp., Coronavirus, CMV, Dengue virus, Ebola virus, EBV, Hepatitis virus, Herpes virus, HIV, Influenza virus, Measles virus, Mumps virus, Papillomavirus, pox virus, polio virus, rabies virus, RSV, West Nile virus, Yellow Fever virus, Aspergillus spp., Blastomyces spp., Candida spp., Coccidioides spp., Cryptococcus spp., Histoplasma spp., Coccidia spp., Cryptosporidum spp., Entamoeba spp., Giardia spp., Leshmania spp., Plasmodium spp., Schistosoma spp., Toxoplasma spp., Trichinella spp., and Trypanosoma spp.

37. The method of claim 37 wherein the infectious agent is selected from the group consisting of B. anthracis, B. brochiseptica, B. parapertussis, B. pertussis, B. burgdorferi, C. trachomatis, Clostridium botulinum, C. diphtheria, E. coli, H. influenzae, H. pylori, M. tubercolosis, N. meningitidis, N. gonnorhoeae, S. entiriditis, S. typhi, Shigella flexneri, S. aureus, S. pneumoniae, V. cholerea, Hepatitis A, Hepatitis B, Hepatitis C, Hepatitis D, Hepatitis E, vaccinia virus, smallpox virus, E. histolytica, Giardia lamblia, and Toxoplasma gondii.

38. The method of claim 35 wherein the cancer is selected from the group consisting of cancer of the colon, rectum, breast, skin, lung, brain, and blood.

39. The method of claim 35 wherein the recombinant virus comprises within its genome a nucleic acid encoding a tumor antigen.

40. The method of claim 39 wherein the tumor antigen is selected from the group consisting of gp100, MART-1/Melan A, gp75 (TRP-1), tyrosinase, melanoma proteoglycan, a MAGE family antigen, MAGE-1, MAGE-2, MAGE-3, MAGE-4, MAGE-6, MAGE-12, a BAGE family antigen, a GAGE family antigens, GAGE-1, GAGE-2, a RAGE family antigen, RAGE-1, N-acetylglucosaminyltransferase-V, p15, β-catenin, MUM-1, cyclin dependent kinase-4, p21-ras, BCR-abl, p53, p185 HER2/neu, epidermal growth factor receptor, carcinoembryonic antigen (CEA), a carcinoma-associated mutated mucin, a MUC-1 gene products, an Epstein-Barr Virus EBNA gene product, papillomavirus E7, papillomavirus E6, prostate specific antigen (PSA), prostate specific membrane antigen (PSMA), an idiotypic antigen, KSA, kinesin 2, HIP-55, TGF β-1 anti-apoptotic factor, tumor protein D52, H1FT, NY-BR-1, NY-BR-62, NY-BR-75, NY-BR-85, NY-BR-87, NY-BR-96, BCY1, BFA4, BCY3, BCZ4, a fragment thereof, and a derivative thereof.
